(19) United States
(12) Patent Application Publication
(10) Pub. No.: US 2024/0371538 A1
(43) Pub. Date: Nov. 7, 2024

(54) 3D PRINTING OF SOLID-STATE PHANTOMS

(71) Applicant: UNIVERSITÄT BERN, Bern (CH)

(72) Inventors: Michael HENTSCHEL, Niederwangen (CH); Tilman LÄPPCHEN, Wabern (CH); Lorenz Paul MEIER, Zürich (CH); George Amadeus PRENOSIL, Herbetswil (CH)

(73) Assignee: UNIVERSITÄT BERN, Bern (CH)

(21) Appl. No.: 18/565,545

(22) PCT Filed: May 30, 2022
(86) PCT No.: PCT/EP2022/064650

§ 371 Date: Nov. 30, 2023

(30) Foreign Application Priority Data
Jun. 1, 2021 (EP) 21177237.1

(51) Int. Cl. G21G 4/08; A61K 51/06; A61K 51/12; B33Y 10/00; B33Y 80/00; C08F 2/06; C08F 2/48; C08K 5/56

(52) U.S. Cl. CPC G21G 4/08; A61K 51/06; A61K 51/121; C08F 2/06; C08F 2/48; C08K 5/56; A61K 2123/00; B33Y 10/00; B33Y 80/00

(57) ABSTRACT

The present invention relates to a method for preparing a radioactive polymerizable solution and the solution per se. The solution is prepared by using a cationic radionuclide with a half-life >5 d that is dissolved in an aqueous solution. The radionuclide is extracted into an organic solvent or monomer solution by using a lipophilic ligand for forming a radioactive complex. The ligand is characterized by a ≥2:1 ratio of carbon and/or silicon atoms to specific heteroatoms. If an organic solvent is used for extraction, the organic phase obtained is subsequently diluted in a monomer solution to obtain the radioactive polymerizable solution. The invention further relates to the use of said solution in a method for preparing a radioactive object such as a phantom for medical imaging and the object per se.

The present invention relates to a method for preparing a radioactive polymerizable solution, the solution per se, a method for preparing a radioactive object, particularly by using the radioactive polymerizable solution, and a radioactive object such as a phantom for medical imaging that is obtained by performing said methods.

### BACKGROUND OF THE INVENTION

Positron emission tomography in combination with computed tomography (PET/CT) quantitatively images metabolic processes. Clinical routine as well as clinical trials rely on verified and calibrated PET/CT systems for the making of reproducible and comparable imaging data. Therefore, acceptance of newly installed PET/CT systems, as well as quality assurance (QA) in nuclear medicine and multicentre clinical trials (MCTs) [1] dictate the testing of clinical PET/CT systems [2].

Such testing as well as periodical quality control (QC) of PET/CT systems usually involve phantoms with clearly defined positron emitter distributions. Even though long-lived solid-state phantoms exist—usually homogenous cylinders for daily QA—most phantoms are a set of voids fillable with liquid radionuclides from clinical use. For example, image quality testing according to the National Electrical Manufacturers Association (NEMA) protocol [3, 4] or compliance tests according to the EARL PET/CT accreditation program [5, 6] mandate the use of fillable spheres situated within a (liquid) background compartment. However, such phantoms provide artefacts due to the non-radioactive walls of the fillable spheres, and they are prone to filling errors (radioisotope concentration of the filling solution, air bubbles, etc.) [7].

With roughly 271 d half-life, a phantom containing germanium-68 in solid-state improves reproducibility of repeated measurements [8] and offers safer radiation handling [9], compared to phantoms filled with aqueous solutions of fluorine-18, gallium-68 or even germanium-68. The long half-life of germanium-68 aids also in the cross-calibration of PET/CT systems in multicentre studies [8, 10]. Furthermore, lower regulatory requirements attached to the transport of solid-state radioactive sources [11] facilitate the exchange of standardized solid-state phantoms within a multicentre consortium. Despite the many advantages, solid-state phantoms have not yet found widespread application, presumably due to the lack of safe and inexpensive methods for their production.

Additive manufacturing holds the promise of supplying solid-state phantoms of arbitrary shape at reasonable cost. While fillable 3D printed phantoms have been around since 2014 [12], direct printing of hot phantoms would avoid any subsequent filling, offer constant quality of their radioactive properties considering the radioisotope half-life during the phantom lifetime and thus, tests are comparable as well as it avoids cold wall artefacts.

Once setup, 3D printing is remarkably simple and cost-effective, but the usually slow printing progress restricts its use to medium- to long-lived radionuclides, such as germanium-68, the mother isotope of the widely used positron emitter gallium-68. However, 3D printing with ⁶⁸Ge-containing liquid resin monomers for the use in PET/CT involves several difficulties, e.g. homogeneous distribution within the final phantom, and hazards. Most importantly, care must be taken to avoid formation and release of volatile radioactive germanium halides during handling and printing, as well as during the whole lifetime of the solid phantom. Clearly, simple mixing of commercially available aqueous ⁶⁸Ge-solutions in dilute hydrochloric acid with hydrophobic monomers for 3D-printing will result in a heterogeneous emulsion since the aqueous phase is immiscible with both the monomers and polymers and inherently poses the risk of radioactive leakage and radioactive contamination due to phase separation and diffusion of the ionic, unbound radioisotope over time. While this may be only a minor issue when fabricating phantoms with short-lived radionuclides such as technetium-99m, it is not acceptable when employing longer-lived radionuclides such as germanium-68, where phase separation together with diffusion instability of volatile ⁶⁸Ge-halide species can promote latent heterogeneity and release of radioactivity over time.

Hitherto, no readily applicable method exists to transfer germanium-68 from aqueous solutions into organic liquid resin building materials in the form of stable hydrophobic complexes of germanium-68, which ensure tight immobilization in the printed phantoms for a long time without release of radioactivity. Our enabling technology bridges the gap, which has so far prevented additive manufacturing of long-lived phantoms with stereolithography and other 3D-printing technologies.

Providing inexpensive, reusable and safe to handle PET/CT phantoms of arbitrary shapes and sizes will simplify QC, characterization of PET/CT imaging properties [13], and performance harmonization of PET/CT [14, 15] systems in daily routine as well as in clinical studies. When printing with multiple printer heads, not only can a phantom's shape be adapted to a specific scientific or clinical demand but also its activity distribution can be adjusted accordingly [16]. However, tight incorporation and homogenous distribution of the radionuclides into the printing resin is key to dispense with cold phantom walls or with a thick protective coating and thus to eliminate any wall-related bias originating from activity spill-in and spill-out [17]. Institutions carrying out MCTs will find the inventive method useful to reduce handling-induced variability of a calibrating phantom in their PET/CT

measurements for comparability [18], while using phantoms with an improved physiological analogy.

Thus, such phantoms require a safe way to incorporate long-lived cationic radionuclides suited for imaging into polymers that can be used for this purpose. We initially tested the concept with the short-lived cationic radionuclide technetium-99 (t ½˜6 h) and identified trioctylphosphine as a suitable complex-forming phase transfer agent, which proved highly effective for the transfer of technetium-99 to an organic solvent, which could then be mixed

with the organic acrylate-based liquid resin monomers for 3D printing [23]. However, subsequent experiments with longer-lived cationic radionuclides showed that adaptions of the results with technetium-99m to other cationic radionuclides were not feasible. The ligand trioctylphosphine turned out to work for technetium-99m only but not generally for other cationic radionuclides.

Therefore, novel procedures needed to be developed, which in many cases require the addition of both a complex-forming anionic lipophilic ligand and another additive (typically a base or a suitable cation or anion) acting as an auxiliary ligand and/or for charge compensation. The general characteristics of these novel procedures are described in this patent, and specific examples are provided for the transfer of germanium-68, cobalt-57, and lutetium-177 from commercially available aqueous solutions acid into an organic phase, which is miscible with acrylate-based liquid resin monomers for 3D-printing.

The present invention aims to produce an easy to handle and long-lived, wall-less, solid-state PET phantom with advanced source homogeneity and maximally safe handling characteristics. The phantoms of the present invention are characterized by stable complexation within the polymer matrix to achieve reliable immobilization of the radionuclide and to avoid leakage.

Complexation and phase transfer of the radionuclides to the hydrophobic monomer was achieved using a general approach of a hydrophobic anionic ligand for chelation of the radioactive cation, if necessary in combination with a pH modifying hydrophobic additive to achieve neutral overall ionic charge of the hydrophobic complex that will distribute predominantly in the hydrophobic phase.

Based on the above-mentioned state of the art, the objective of the present invention is to provide means and methods to prepare a radioactive polymerizable solution that is suitable for preparing a radioactive object such as a phantom. This objective is attained by the subject-matter of the independent claims of the present specification, with further advantageous embodiments described in the dependent claims, examples, figures and general description of this specification.

### SUMMARY OF THE INVENTION

A first aspect of the invention relates to a method for preparing a radioactive polymerizable solution comprising the steps of
a) providing
   an aqueous radionuclide solution comprising a cationic radionuclide with a half-life >5 d,
   a complex-forming lipophilic ligand, wherein
      the complex forming lipophilic ligand comprises one or more carbon atoms and/or one or more silicon atoms, and one or more heteroatoms, wherein at least one heteroatom is selected from oxygen, nitrogen, sulfur, phosphorus, selenium and arsenic, and wherein the ratio of carbon atoms and/or silicon atoms to heteroatoms is ≥2:1, particularly ≥2.5:1, more particularly ≥3:1, wherein the heteroatoms that are considered for the ratio are selected from oxygen, nitrogen, sulfur, phosphorus, selenium and arsenic, and
      the mass of the complex-forming lipophilic is <5000 g/mol, particularly <1500 g/mol,
   optionally, an additive, wherein the additive is a base or a suitable cation or anion acting as an auxiliary ligand and/or for charge compensation,
   a monomer solution comprising one or more monomers selected from an acrylate and a methacrylate, wherein the monomer solution is non-miscible with said aqueous radionuclide solution,
   optionally, an organic solvent,
b) mixing the aqueous radionuclide solution with
   the complex forming lipophilic ligand, and
   optionally the additive, and
   the monomer solution or the organic solvent yielding an aqueous phase and a radioactive organic phase,
c) separating and collecting the radioactive organic phase in case of using the monomer solution in step (b), or separating and collecting the radioactive organic phase in case of using the organic solvent in step (b) and adding said monomer solution to the radioactive organic phase.

A second aspect of the invention relates to a radioactive polymerizable solution, particularly prepared according to the method according to the first aspect of the invention, comprising a complex dissolved in a monomer solution comprising one or more monomers selected from an acrylate and a methacrylate, wherein the complex comprises.
a cationic radionuclide with a half-life >5 d, and
a complex-forming lipophilic ligand, wherein
the complex forming lipophilic ligand comprises one or more carbon atoms and/or one or more silicon atoms, and one or more heteroatoms, wherein at least one heteroatom is selected from oxygen, nitrogen, sulfur, phosphorus, selenium and arsenic, and wherein the ratio of carbon atoms and/or silicon atoms to heteroatoms is 2:1, particularly 2.5:1, more particularly 3:1, wherein the heteroatoms that are considered for the ratio are selected from oxygen, nitrogen, sulfur, phosphorus, selenium and arsenic, and
the mass of the complex-forming lipophilic ligand is <5000 g/mol, particularly <1500 g/mol,
optionally an additive, wherein the additive is a base or suitable cation or anion.

A third aspect of the invention relates to a method for preparing a radioactive object. The method comprises the steps of
a) providing a radioactive polymerizable solution prepared according to the method according to the first aspect of the invention, or the radioactive polymerizable solution according to the second aspect of the invention,
b) adding an initiator, particularly a photoinitiator, more particularly phenylbis(2,4,6-trimethylbenzoyl)phosphine oxide,
c) polymerization by using light or UV light.

A fourth aspect of the invention relates to a radioactive object, particularly prepared according to the method according to the third aspect of the invention. The radioactive object comprises a complex distributed in a polymer network comprising one or more polymers selected from an acrylate polymer and a methacrylate polymer, wherein the complex comprises
a cationic radionuclide with a half-life >5 d, and
a complex-forming lipophilic ligand, wherein
the complex-forming lipophilic ligand comprises one or more carbon atoms and/or one or more silicon atoms, and one or more heteroatoms, wherein at least one heteroatom is selected from oxygen, nitrogen, sulfur, phosphorus, selenium and arsenic, and wherein the ratio of carbon atoms and/or silicon atoms to heteroatoms is 2:1, particularly 2.5:1, wherein the heteroatoms that are considered for the ratio are selected from oxygen, nitrogen, sulfur, phosphorus, selenium and arsenic, and
the mass of the complex-forming lipophilic ligand is <5000 g/mol, particularly <1500 g/mol,
optionally an additive, wherein the additive is a base or suitable cation or anion.

### DESCRIPTION OF THE INVENTION

### Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art.

The term phantom in the context of the present specification relates to an object that is scanned or imaged in the field of medical imaging to evaluate, analyse, and tune the performance of an imaging device, particularly an imaging device for use in SPECT (single-photon emission computed tomography) or PET (positron emission tomography) (https://en.wikipedia.org/wiki/Imaging_phantom-cite_note-1). The phantom may be of arbitrary shape. Non-limiting examples for suitable shapes are a sphere, a cylinder or a cuboid. Phantoms according to the invention are characterized by a defined radioactivity distribution, particularly a uniform radioactivity distribution, and they do not need to comprise cold interfaces between different phantom parts or cold walls. Furthermore, phantoms according to the invention are solid-state phantoms.

In the context of the present invention, the term derivative relates to a compound that is substituted by one or more moieties selected from alkyl, phenyl, ether, ester, alcohol, particularly alkyl, phenyl, ether, ester. For example, a derivative of tartaric acid is tartaric acid substituted by an alkyl moiety.

### DETAILED DESCRIPTION

A first aspect of the invention relates to a method for preparing a radioactive polymerizable solution comprising the steps of
a) providing
   an aqueous radionuclide solution comprising a cationic radionuclide with a half-life >5 d,
   a complex-forming lipophilic ligand, wherein
      the complex forming lipophilic ligand comprises one or more carbon atoms and/or one or more silicon atoms, and one or more heteroatoms, wherein at least one heteroatom is selected from oxygen, nitrogen, sulfur, phosphorus, selenium and arsenic, and wherein the ratio of carbon atoms and/or silicon atoms to heteroatoms is ≥2:1, particularly ≥2.5:1, more particularly ≥3:1, wherein the heteroatoms that are considered for the ratio are selected from oxygen, nitrogen, sulfur, phosphorus, selenium and arsenic, and
      the mass of the complex-forming lipophilic is <5000 g/mol, particularly <1500 g/mol,
   optionally, an additive, wherein the additive is a base or a suitable cation or anion acting as an auxiliary ligand and/or for charge compensation,
   a monomer solution comprising one or more monomers selected from an acrylate and a methacrylate, wherein the monomer solution is non-miscible with said aqueous radionuclide solution,
   optionally, an organic solvent,
b) mixing the aqueous radionuclide solution with
   the complex forming lipophilic ligand, and
   optionally the additive, and
   the monomer solution or the organic solvent yielding an aqueous phase and a radioactive organic phase,
c) separating and collecting the radioactive organic phase in case of using the monomer solution in step (b), or separating and collecting the radioactive organic phase in case of using the organic solvent in step (b) and adding said monomer solution to the radioactive organic phase.

The method according to the first aspect of the invention provides a radioactive polymerizable solution that is suitable for 3D printing. Particularly in the field of medical imaging, there is a need for easy to handle and long-lived solid-state phantoms wherein the radioactivity is evenly distributed within the phantom.

To obtain a long-lived solid-state phantom that allows improved reproducibility and accuracy of measurements such as PET and SPECT measurements, the radionuclide should have a half-life of more than 5 days. For instance, germanium-68 with a half-life of 271 days is a suitable radionuclide for phantoms used in PET applications.

Radionuclides are commercially available as aqueous solutions such as cationic germanium-68 diluted in aqueous hydrochloric acid. The aqueous solution is not miscible with organic liquid 3D building material, i.e. polymerizable monomers suitable for 3D printing. Therefore, the present method makes use of a complex-forming lipophilic ligand. The complex-forming lipophilic ligand is characterized by a ratio of carbon and/or silicon atoms to heteroatoms ≥2:1, particularly ≥2.5:1, more particularly ≥3:1, which allows for complex formation with a cationic radionuclide and phase transfer of the complex into an organic solvent or a monomer solution. The hydrophobic complexes are stable.

In certain embodiments, the heteroatoms that are considered for the ratio are selected from oxygen, nitrogen, sulfur, phosphorus and selenium, particularly oxygen, nitrogen, sulfur and phosphorus.

In certain embodiments, a base or a suitable anion or cation is added as an auxiliary ligand and/or for charge compensation to yield an uncharged complex. For example, the extraction of germanium-68 requires the presence of both a complexing agent such as dodecyl gallate as well as an additive such as the phase transfer agent tributylamine.

When performing the present method, the aqueous radionuclide solution is mixed with a complex forming lipophilic ligand, optionally an additive is added as an auxiliary ligand and/or for charge compensation to yield an uncharged complex, and the monomer solution suitable for 3D printing is added or an organic solvent is used instead of the monomer solution.

In the combined complexation and extraction procedure, the complexed radionuclide is transferred to the organic phase, i.e. to the monomer solution or the organic solvent, and subsequently, the aqueous phase and the organic phase are separated. The complexation of the radioisotope and phase transfer is an efficient equilibrium-controlled process that leads to a homogenous distribution of the complexes within the hydrophobic phase.

Simple mixing of commercially available aqueous radionuclide solutions with hydrophobic monomers for 3D printing would result in an emulsion and inherently poses the risk of radioactive leakage and radioactive contamination due to phase separation and diffusion of the ionic, unbound isotope over time. Phase separation together with diffusion instability of uncomplexed and potentially volatile radioactive species can promote latent heterogeneity and release of radioactivity over time. The present invention avoids the release of volatile radioactive compounds such as radioactive germanium halides by the formation of stable complexes. This allows safe handling of the radioactive polymerizable solution as well as of the printed object.

When the monomer solution was added in the mixing step, the collected organic phase can directly be used as radioactive polymerizable solution in a 3D printing process. If required, the solution may be diluted by adding monomer solution to adjust the desired radioactivity concentration.

When an organic solvent was added in the mixing step, the collected organic phase is diluted in a monomer solution that is suitable for use in 3D printing. Similarly, the desired radioactivity concentration can be adjusted by adding a suitable amount of monomer solution.

This two-step approach (first using an organic solvent and subsequent dilution in a suitable monomer solution) is particularly performed when the radionuclide is germanium-68.

In certain embodiments, the method for preparing a radioactive polymerizable solution comprises the steps of
a) providing
   an aqueous radionuclide solution comprising a cationic radionuclide with a half-life >5 d,
   a complex-forming lipophilic ligand, wherein
      the complex forming lipophilic ligand comprises one or more carbon atoms and/or one or more silicon atoms, and one or more heteroatoms, wherein at least one heteroatom is selected from oxygen, nitrogen, sulfur, phosphorus, selenium and arsenic, and wherein the ratio of carbon atoms and/or silicon atoms to heteroatoms is ≥2:1, particularly ≥2.5:1, more particularly ≥3:1, wherein the heteroatoms that are considered for the ratio are selected from oxygen, nitrogen, sulfur, phosphorus, selenium and arsenic, and
      the mass of the complex-forming lipophilic ligand is <5000 g/mol, particularly <1500 g/mol,
   optionally, an additive, wherein the additive is a base or a suitable cation or anion acting as an auxiliary ligand and/or for charge compensation,
   a monomer solution comprising one or more monomers selected from an acrylate and a methacrylate, wherein the monomer solution is non-miscible with said aqueous radionuclide solution,
   optionally, an organic solvent,
b) mixing the aqueous radionuclide solution with
   the complex forming lipophilic ligand, and
   optionally the additive, wherein particularly an additive is added if the radionuclide is germanium-68, and
   the organic solvent yielding an aqueous phase and a radioactive organic phase,
c) separating and collecting the radioactive organic phase and adding said monomer solution to the radioactive organic phase.

To increase the yield, the extraction of the cationic radionuclide from the aqueous radionuclide solution may be performed repeatedly. Step (b) and (c) as described above are repeated wherein the aqueous phase from the previous performance is used as aqueous radionuclide solution.

In certain embodiments, steps (b) and (c) are performed repeatedly.

In certain embodiments, the cationic radionuclide is a cation of Ge, Na, Co or Lu.

In certain embodiments, the cationic radionuclide is a cation of ⁶⁸Ge, ²²Na, ⁵⁷Co or ¹⁷⁷Lu.

The aqueous radionuclide solution may comprise only one type of cations, e.g. only [⁶⁸Ge]Ge⁴⁺, or a mixture of different cations, e.g. [¹⁷⁷Lu]Lu³⁺ and [⁶⁸Ge]Ge⁴⁺.

The aqueous radionuclide solution may contain the cationic radionuclide in the form of a dissociated salt, potentially also as hydroxo-species and other complexes in solution. Cationic radionuclides are commercially available in different solutions. For examples, [⁶⁸Ge]Ge⁴⁺ may be present in the radioactive aqueous solution as dissolved halide such as [⁶⁸Ge]Ge chloride.

In certain embodiments, the cationic radionuclide in the radioactive aqueous solution is part of a compound selected from a halide, particularly chloride, a nitrate, a hydroxide, a phosphate, a sulfate, a cation counterbalanced by an amino acid anion, particularly aspartate, an acetate, a lactate, a pyruvate, a bicarbonate.

In certain embodiments, the cationic radionuclide is in form of a salt.

In certain embodiments, the cationic radionuclide is selected from [⁶⁸Ge]Ge-salt, [⁶⁸Ge]Ge-hydroxide, [²²Na]Na-salt, [²²Na]Na-hydroxide, [⁵⁷Co]Co-salt, [¹⁷⁷Lu]Lu-salt. In aqueous solutions, the salts are usually dissociated and the cationic radionuclide may be associated with multiple counter ions, including also formation of hydroxo-species.

In certain embodiments, the cationic radionuclide is selected from [⁶⁸Ge]Ge-salt, [²²Na]Na-salt, [⁵⁷Co]Co-salt, [¹⁷⁷Lu]Lu-salt.

In certain embodiments, the salt is a chloride or nitrate. Non-limiting examples are [⁶⁸Ge]Ge chloride or [⁶⁸Ge]Ge nitrate.

In certain embodiments, the salt is a chloride.

Suitable cationic radionuclides are often forming insoluble colloidal hydroxides at neutral pH. The solubility of cationic radionuclides in aqueous solutions can be increased under acidic or basic conditions. Particularly radioactive cations of Ge, Co and Lu are soluble under acidic conditions while [²²Na]Na⁺is soluble in acidic, neutral or alkaline solutions, particularly neutral or alkaline solutions.

In certain embodiments, the pH of the radionuclide solution is <7 if the radionuclide is a cation of Na, Ge, Co or Lu, and/or the pH of the radionuclide solution is >7 if the radionuclide is a cation of Na.

In certain embodiments, the pH of the radionuclide solution is <7 if the radionuclide is a cation of Ge, Co or Lu, and/or the pH of the radionuclide solution is >7, particularly >7, if the radionuclide is a cation of Na.

In certain embodiments, the pH of the radionuclide solution is <7, particularly <5, more particularly <3, even more particularly <2.

In certain embodiments, the aqueous radionuclide solution comprises an acid, particularly hydrochloric acid.

In certain embodiments, the aqueous radionuclide solution comprises 0.01 to 1.0 M, particularly 0.01 to 0.1 M HCl, and even more particularly 0.05 M HCl.

Not only the solubility of the cationic radionuclide in the aqueous solution but also the distribution of the complex-forming lipophilic ligand in the aqueous and organic phase is pH dependent. The pH-dependent distribution of a compound between the aqueous and the organic phase is generally described by its distribution coefficient D, which is the ratio of the sum of the concentration of all species of the compound in 1-octanol to the sum of the concentration of all species of the compound in the aqueous phase at a particular pH. The pH-dependent distribution coefficient D_{pH} is generally reported as its logarithm, logD_{pH}. For various ligands, logD_{pH}-values are either known or can be determined by those skilled in the art, or can be estimated using methods described in the literature [19, 20] and implemented in commercially available computer programs building, e.g. “ACD/Labs” (https://www.acdlabs.com/), “ChemAxon” (https://chemaxon.com/), and others. Such tabulated or calculated logD_{pH}-values can be used for pre-selection of suitable complex-forming ligands. The final suitability of a complex-forming ligand, however, is determined by the distribution of its radiometal-complex, if applicable including also the suitable additive mentioned in step a) and b). Under the specific conditions applied in the extraction, the distribution coefficient of this radiometal-complex between the organic phase and the water phase should be at least >5:1, particularly for monovalent cations such as Na.

In certain embodiments, the distribution coefficient of this radiometal-complex between the organic phase and the water phase, particularly for multivalent cations such as Ge, Co, Lu, should be at least >10:1.

In certain embodiments, the distribution coefficient of this radiometal-complex between the organic phase and the water phase, particularly for multivalent cations such as Ge, Co, Lu, should be at least >30:1.

In certain embodiments, the distribution coefficient of this radiometal-complex between the organic phase and the water phase should be at least >1000:1.

In certain embodiments, the distribution coefficient of this radiometal-complex between the organic phase and the water phase should be at least >10000:1.

The complex-forming ligand is composed of carbon and/or silicon atoms as well as of one or more heteroatoms. The composition of atoms determines the lipophilicity of the complex-forming ligand. Apart from the heteroatoms that are decisive for determining the ratio of carbon and/or silicon atoms to heteroatoms as described above, the complex-forming ligand may comprise further heteroatoms such as boron or halogens.

In certain embodiments, the one or more heteroatoms in the complex-forming lipophilic ligand are independently from each other selected from oxygen, nitrogen, sulfur, phosphorus, selenium, arsenic, boron and a halogen.

In certain embodiments, the complex-forming lipophilic ligand is selected from a gallate, particularly an alkyl gallate, pyrocatechol, a di- or tricarboxylic acid, tannin or a derivative thereof, 8-hydroxyquinoline or a derivative thereof, a crown-ether or a derivative thereof, a cryptand or a derivative thereof, a podand or a derivative thereof, a spherand or a derivative thereof, a calixarene or a derivative thereof, trialkylphosphine, a thiol or a derivative thereof, a thioether or a derivative thereof, mono-, di-, or tri-alkyl-DTPA or derivatives thereof containing aryl, heteroaryl and/or other functionalities at any position in one or more of the alkyl chains, or mono-, di-, and tri-alkyl-DOTA or derivatives thereof containing aryl, heteroaryl and/or other functionalities at any position in one or more of the alkyl chains.

In certain embodiments, the complex-forming lipophilic ligand is selected from a gallate, particularly an alkyl gallate, pyrocatechol, a di- or tricarboxylic acid, tannin or a derivative thereof, 8-hydroxyquinoline or a derivative thereof, a crown-ether or a derivative thereof, a cryptand or a derivative thereof, a podand or a derivative thereof, a spherand or a derivative thereof, a calixarene or a derivative thereof, a thiol or a derivative thereof, a thioether or a derivative thereof, mono-, di-, or tri-alkyl-DTPA or derivatives thereof containing aryl, heteroaryl and/or other functionalities at any position in one or more of the alkyl chains, or mono-, di-, and tri-alkyl-DOTA or derivatives thereof containing aryl, heteroaryl and/or other functionalities at any position in one or more of the alkyl chains.

In certain embodiments, the complex-forming lipophilic ligand is a gallate, particularly an alkyl gallate.

In certain embodiments, in case of a cation of ⁶⁸Ge, the complex forming lipophilic ligand is selected from a gallate, particularly an alkyl gallate, pyrocatechol, a di-or tricarboxylic acid, tannin or a derivative thereof, 8-hydroxyquinoline or a derivative thereof, and/or,

in case of a cation of ²²Na, the complex forming lipophilic ligand is selected from a crown ether or a derivative thereof, particularly a mono anionic crown ether derivative, and/or,

in case of a cation of ⁵⁷Co, the complex forming lipophilic ligand is selected from trialkylphosphine, a thioether or a derivative thereof, mono-, di-, or tri-alkyl-DTPA or a derivative thereof, mono-, di-, or tri-alkyl-DOTA or a derivative thereof or a mixture thereof, and/or,

in case of a cation of ¹⁷⁷Lu, the complex forming lipophilic ligand is selected from mono-, di-, or tri-alkyl-DTPA or a derivative thereof or mono-, di-, or tri-alkyl-DOTA or a derivative thereof.

In certain embodiments, in case of a cation of ⁶⁸Ge, the complex forming lipophilic ligand is selected from a gallate, particularly an alkyl gallate, pyrocatechol, a di- or tricarboxylic acid, tannin or a derivative thereof, 8-hydroxyquinoline or a derivative thereof, and/or,

in case of a cation of ²²Na, the complex forming lipophilic ligand is selected from a crown ether or a derivative thereof, particularly a mono anionic crown ether derivative, and/or,

in case of a cation of ⁵⁷Co, the complex forming lipophilic ligand is selected from a thioether or a derivative thereof, mono-, di-, or tri-alkyl-DTPA or a derivative thereof, mono-, di-, or tri-alkyl-DOTA or a derivative thereof or a mixture thereof, and/or,

in case of a cation of ¹⁷⁷Lu, the complex forming lipophilic ligand is selected from mono-, di-, or tri-alkyl-DTPA or a derivative thereof or mono-, di-, or tri-alkyl-DOTA or a derivative thereof.

In certain embodiments, the gallate is a C₃₋₂₀-alkyl gallate, particularly a C₆₋₁₈-alkyl gallate, more particularly dodecyl gallate.

In certain embodiments, the dicarboxylic acid is a compound of formula (I),

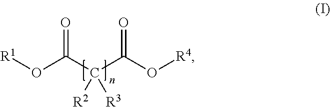

wherein R¹, and R⁴ are independently selected from H, a linear or branched alkyl, wherein the alkyl is optionally substituted by —OH, halogen, aryl, heteroaryl

R² and R³ are independently selected from H, —OH, a linear or branched alkyl, wherein the alkyl is optionally substituted by —OH, halogen, aryl, heteroaryl

n is an integer between 0 and 6, particularly between 0 and 3,

wherein the moieties R¹, R², R³ and R⁴ are selected in such a way that the ratio of C atoms to heteroatoms is as described above.

For example, a compound characterized by R¹ and R⁴ being C₃-alkyl, n=1, R² and R³ being H comprises in total 9 C atoms and 4 heteroatoms (oxygen). Thus, the ratio of C atoms to heteroatoms is larger than 2:1.

If n is ≥2, R² and R³ may vary between different Cₙ. For instance, if n is 2, R² bound to the first C atom may be H, R³ bound the first C atom may be —OH, R² bound to the second C atom may be H and R³ bound to the second C atom may be an alkyl moiety.

Non-limiting examples for suitable ligands are derivatives of oxalic acid or tartaric acid.

In certain embodiments, n is 0, 1 or 2.

In certain embodiments, the tricarboxylic acid is a compound of formula (II),

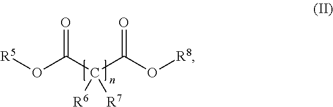

wherein

R⁵ and R⁸ are independently selected from H, a linear or branched alkyl, wherein the alkyl is optionally substituted by —OH, halogen, aryl, heteroaryl

R⁶ and R⁷ are independently selected from H, —OH, —COOH, a linear or branched alkyl, wherein the alkyl is optionally substituted by —OH, —COOH, halogen, aryl, heteroaryl

m is an integer between 1 and 6, particularly between 1 and 4,

wherein the moieties R⁵, R⁶, R⁷ and R⁸ are selected in such a way that the ratio of C atoms to heteroatoms is as described above, and at least one of the moieties R⁶ and R⁷ comprises a carboxylic acid moiety.

If m is ≥2, R⁶ and R⁷ may vary between different Cₘ. For instance, if m is 2, R⁶ bound to the first C atom may be H, R⁷ bound the first C atom may be —COOH, R⁶ bound to the second C atom may be H and R⁷ bound to the second C atom may be an alkyl moiety.

A non-limiting example for suitable ligand is a derivative of citric acid.

In certain embodiments, m is 3.

Upon polymerization of the monomers during 3D printing, the radioactive complex is trapped in the polymer network. To further immobilize the radioactive complex, the lipophilic ligand may comprise an acrylate or methacrylate moiety. Such moiety would allow covalent bond formation between the lipophilic ligand of the radioactive complex and a monomer of the polymer network due to copolymerization during 3D printing.

In certain embodiments, the complex forming ligand is substituted by one or more acrylate moieties and/or one or more methacrylate moieties.

Particularly if the complex formed by the ligand and the cationic radionuclide is negatively or positively charged, a base or a suitable anion or cation is added as an auxiliary ligand and/or for charge compensation to yield an uncharged complex.

In certain embodiments, the additive is selected from a hydrophobic amine, particularly a mono-, di-, or trialkylamine, the corresponding tetraalkylammonium salts, a triflate, a mesylate, a tosylate, a benzoate, a salicylate, a perchlorate, a tetrafluoroborate, tetrafluoro carboxylate, an alkyl sulfonate, an alkyl phosphonate.

In certain embodiments, the additive is selected from a hydrophobic amine, particularly a mono-, di-, or trialkylamine, the corresponding tetraalkylammonium salts, a triflate, a mesylate, a tosylate, a benzoate, a salicylate, a perchlorate, a tetrafluoroborate, tetrafluoro carboxylate, an alkyl sulfonate, an alkyl phosphonate, wherein the alkyl moiety comprises at least 3 C atoms.

In certain embodiments, the additive is a hydrophobic amine, particularly a mono-, di-, or trialkylamine. In certain embodiments, the additive is a trialkylamine.

In certain embodiments, the linear or branched alkyl moieties of the mono- di- or trialkylamine each comprise 2 to 18 C atoms.

The alkyl moieties may be linear or branched. In case of di- or trialkylamines, the alkyl moieties may be identical or vary. For instance, a suitable trialkylamine with identical alkyl moieties is tributylamine (N,N-dibutylbutan-1-amine, CAS No. 102-82-9). A non-limiting example for a trialkylamine with varying alkyl moieties is N, N-diisopropylethylamine (N -ethyl-N-(propan-2-yl) propan-2-amine, CAS No. 7087-68-5).

In certain embodiments, the additive is N,N-diisopropylethylamine or tributylamine.

In certain embodiments, the additive is tributylamine.

A trialkylamine having alkyl moieties that each comprise 18 C atoms has in total 54 C atoms.

In certain embodiments, the total amount of C atoms of the trialkylamine is ≤54, particularly between 6 and 24.

Similar to the ligands described above, also the additive may comprise an acrylate or methacrylate moiety for copolymerization.

In certain embodiments, the additive is substituted by one or more acrylate moieties and/or one or more methacrylate moieties.

For facilitating the phase transfer of [⁶⁸Ge]Ge⁴⁺ containing complexes, an additive such as tributylamine is added in step (b).

In certain embodiments, the additive is used in step (b) if the radionuclide is a cation of ⁶⁸Ge.

To allow the formation of a polymer network upon 3D printing, the monomers of the monomer solution comprise one or more acrylate and/or methacrylate moieties.

In certain embodiments, the acrylate of the monomer solution is selected from a mono-acrylate, a di-acrylate and a tri-acrylate, and/or

the methacrylate of the monomer solution is selected from a mono-methacrylate, a di-methacrylate and a tri-methacrylate.

In certain embodiments, the monomer is selected from tricyclodecane dimethanol diacrylate (CAS No. 42594-17-2), tricyclodecane dimethanol di(meth)acrylate (CAS No. 43048-08-4), mono-, di-, or tri-ethylene glycol diacrylate (CAS No. 2274-11-5, CAS No. 4074-88-8, and CAS No. 1680-21-3, respectively), mono-, di-, or tri-ethylene glycol di (meth) acrylate (CAS No. 97-90-5, CAS No. 2358-84-1, and CAS No. 109-16-0, respectively), bisphenol A ethoxylate diacrylate (CAS No. 64401-02-1), bisphenol A ethoxylate dimethacrylate (CAS No. 41637-38-1).

In certain embodiments, the monomer is selected from tricyclodecane dimethanol diacrylate, mono-, di-, or tri-ethylene glycol diacrylate, bisphenol A ethoxylate dimethacrylate.

In certain embodiments, the monomer solution comprises one or more monomers. For example, the monomer solution may comprise a mixture of triethylene glycol diacrylate and tricyclodecane dimethanol diacrylate.

To facilitate phase separation and to allow safe and easy handling during collecting the radioactive organic phase, the radioactive organic phase should be preferably the upper phase and the aqueous phase should be the lower phase. In certain cases, the radioactive organic phase could also be the lower phase, but this situation is less preferred.

In certain embodiments, the monomer solution provides a density difference of >0.05 g/ml to the aqueous solution.

In certain embodiments, the monomer solution has a density ≤0.95 g/mL.

If the monomer solution that is typically used for 3D printing has a high viscosity, the extraction may be performed with an organic solvent that is less viscous. Subsequently, the radioactive organic phase obtained is mixed with the monomer solution prior to 3D printing.

In certain embodiments, the organic solvent provides a density difference of >0.05 g/ml to the aqueous solution.

In certain embodiments, the organic solvent has a density ≤0.95 g/mL.

To enhance polymerization, the organic solvent may comprise an acrylate and/or methacrylate moiety.

In certain embodiments, the organic solvent is selected from an acrylate, a methacrylate and acetate, or a mixture thereof.

In certain embodiments, the organic solvent is selected from an alkylacrylate, alkylmethacrylate and alkylacetate, or a mixture thereof.

In certain embodiments, the organic solvent is selected from a C₂₋₁₂-alkylacrylate, C₂₋₁₂-alkylmethacrylate and a C₂₋₁₂-alkylacetate, or a mixture thereof.

In certain embodiments, the organic solvent is selected from butyl acrylate and butyl acetate.

In certain embodiments, the organic solvent has a boiling point >80° C.

A second aspect of the invention relates to a radioactive polymerizable solution, particularly prepared according to the method according to the first aspect of the invention, comprising a complex dissolved in a monomer solution comprising one or more monomers selected from an acrylate and a methacrylate, wherein the complex comprises
a cationic radionuclide with a half-life >5 d, and
a complex-forming lipophilic ligand, wherein
the complex forming lipophilic ligand comprises one or more carbon atoms and/or one or more silicon atoms, and one or more heteroatoms, wherein at least one heteroatom is selected from oxygen, nitrogen, sulfur, phosphorus, selenium and arsenic, and wherein the ratio of carbon atoms and/or silicon atoms to heteroatoms is ≥2:1, particularly ≥2.5:1, more particularly ≥3:1, wherein the heteroatoms that are considered for the ratio are selected from oxygen, nitrogen, sulfur, phosphorus, selenium and arsenic, and
the mass of the complex-forming lipophilic ligand is <5000 g/mol, particularly <1500 g/mol,
optionally an additive, wherein the additive is a base or suitable cation or anion.

The radioactive polymerizable solution may be used in 3D printing, particularly for producing solid-state phantoms that are characterized by an evenly distributed radioactivity within the phantom.

To allow applications in medical imaging such as PET or SPECT measurements, the phantom needs to pass a certain threshold in radioactivity, which also applied to the radioactive polymerizable solution from which the phantom is produced.

In certain embodiments, the radioactivity concentration of the radioactive polymerizable solution is 0.1 kBq-1 MBq per mL, particularly 1-100 kBq per mL.

The radioactive polymerizable solution may comprise further compounds that are required for 3D printing such as an initiator to start the polymerization reaction. Suitable initiators are known to those of skill in the art.

In certain embodiments, the radioactive polymerizable solution further comprises an initiator, particularly a photoinitiator, more particularly phenylbis(2,4,6-trimethylbenzoyl)phosphine oxide.

Reference is made to the description and embodiments of the first aspect of the invention, particularly with regard to the monomer solution, the cationic radionuclide, the complex forming lipophilic ligand and the additive.

A third aspect of the invention relates to a method for preparing a radioactive object. The method comprises the steps of
a) providing a radioactive polymerizable solution prepared according to the method according to the first aspect of the invention, or the radioactive polymerizable solution according to the second aspect of the invention,
b) adding an initiator, particularly a photoinitiator, more particularly phenylbis (2,4,6-trimethylbenzoyl)phosphine oxide,
c) polymerization by using light or UV light.

As described above, the radioactive polymerizable solution according to the first or second aspect of the invention may be used in a method for preparing a radioactive object such as a phantom for PET or SPECT measurements.

In certain embodiments, the radioactivity concentration of the radioactive polymerizable solution is 0.1 kBq-1 MBq per mL, particularly 1-100 kBq per mL.

The method may be performed by using a 3D printer. Standard 3D printing processes such as stereolithography may be applied to obtain a radioactive object of arbitrary shape. Typically, the radioactive polymerizable solution is filled into a cartridge of a 3D printer and the object is built layer by layer and cured by light or UV light, i.e. a photoinitiator is activated by light or UV light to start the polymerization reaction. The process for incorporation of the isotopes into the polymer matrix is energetically (or enthalpy) controlled, as opposed to entropy controlled. Thus, greater stability is conveyed to the system.

In certain embodiments, a 3D printer is used for performing the method according to the third aspect of the invention.

Reference is made to the description and embodiments of the first and second aspect of the invention, particularly with regard to the monomer solution, the cationic radionuclide, the complex forming lipophilic ligand and the additive.

A fourth aspect of the invention relates to a radioactive object, particularly prepared according to the method according to the third aspect of the invention. The radioactive object comprises a complex distributed in a polymer network comprising one or more polymers selected from an acrylate polymer and a methacrylate polymer, wherein the complex comprises
a cationic radionuclide with a half-life >5 d, and
a complex-forming lipophilic ligand, wherein
the complex-forming lipophilic ligand comprises one or more carbon atoms and/or one or more silicon atoms, and one or more heteroatoms, wherein at least one heteroatom is selected from oxygen, nitrogen, sulfur, phosphorus, selenium and arsenic, and wherein the ratio of carbon atoms and/or silicon atoms to heteroatoms is ≥2:1, particularly ≥2.5:1, more particularly >3:1, wherein the heteroatoms that are considered for the ratio are selected from oxygen, nitrogen, sulfur, phosphorus, selenium and arsenic, and
the mass of the complex-forming lipophilic ligand is <5000 g/mol, particularly <1500 g/mol,
optionally an additive, wherein the additive is a base or suitable cation or anion or anion acting as an auxiliary ligand and/or for charge compensation.

The radioactive object may be a phantom that is useful in medical imaging such as PET or SPECT measurements.

The radioactive object is characterized by radioactive complexes that are evenly distributed within a polymer network. Such phantoms without cold walls are long-lived and easy to handle. Furthermore, improved reproducibility and accuracy of repeated measurements compared to known phantoms can be achieved. The complexed radioisotope is stable incorporated into the rigid polymer matrix which prevents leaching and adds operational safety. Furthermore, the regulatory requirements associated with the transport of solid-state radioactive sources are lower compared to liquid-filled radioactive phantoms. This facilitates the exchange of standardized solid-state phantoms within a multicenter consortium.

In certain embodiments, a plurality of said complexes is evenly distributed within the polymer network.

In certain embodiments, the polymer is selected from tricyclodecane dimethanol diacrylate polymer, tricyclodecane dimethanol di(meth)acrylate polymer, mono-, di-, or tri-ethylene glycol diacrylate polymer, mono-, di-, or tri-ethylene glycol di(meth)acrylate polymer, bisphenol A ethoxylate diacrylate polymer, bisphenol A ethoxylate dimethacrylate polymer, or a mixture thereof.

In certain embodiments, the polymer is selected from tricyclodecane dimethanol diacrylate polymer, mono-, di-, or tri-ethylene glycol diacrylate polymer and bisphenol A ethoxylate dimethacrylate polymer or a mixture thereof.

In certain embodiments, the polymer is a copolymer comprising polymerized triethylene glycol diacrylate and tricyclodecane dimethanol diacrylate.

In certain embodiments, the radioactive object is a phantom for quantitative positron emission tomography (PET) or/and PET/CT and/or quantitative single emission computed tomography (SPECT) or/and SPECT/CT and/or other devices for the quantitative detection of radioactivity.

In certain embodiments, the radioactive object is a phantom for quantitative positron emission tomography (PET) or/and PET/CT and/or quantitative single emission computed tomography (SPECT) or/and SPECT/CT.

The radioactive object may be used for dosimetry calculations and/or for radiation therapy planning and/or demonstration purposes. In certain embodiments, the radioactive object represents, particularly has the shape of, an organ, tumor, another body part or combinations thereof.

Reference is made to the description and embodiments of the first, second and third aspect of the invention, particularly with regard to the monomers that polymerize to form the polymer network, the cationic radionuclide, the complex forming lipophilic ligand and the additive.

The invention is further illustrated by the following examples and figures, from which further embodiments and advantages can be drawn. These examples are meant to illustrate the invention but not to limit its scope.

### DESCRIPTION OF THE FIGURES

FIG. 1 shows photographs taken of the printed (a) and liquid filled sphere (b).

FIG. 2 shows x/z-planes through the centroid of the 3D printed and the liquid filled 8 mL sphere taken from FBP images. The red and green lines mark positions of line profiles, and the black circle marks the real extend of the 8 mL sphere. Below the images are the respective x-, y-, and z-line profiles through the sphere's centroids as seen in the PET images above. The line profiles are integral normalized for better comparability. The green arrow points to the filler neck of the liquid filled sphere.

FIG. 3 shows x/z-planes through the centroid of the 3D printed and the liquid filled 8 mL sphere taken from OSEM images. The red and green lines mark positions of line profiles, and the black circle marks the real extend of the 8 mL sphere. Below the images are the respective x-, y-, and z-line profiles through the sphere's centroids as seen in the PET images above. The line profiles are integral normalized for better comparability. The green arrow point to the filler neck of the liquid filled sphere.

FIG. 4 shows cumulative probability histograms of activity found in PET/CT images of the 8 mL sphere reconstructed with FBP (a) and OSEM (b).

FIG. 5 High-resolution CT scans of non-radioactive (8 ml) printed spheres. Arrows point out voids in the print.

FIG. 6 PET-scan of a 68Ge-sphere phantom according to NEMA specifications in the presence of a fast decaying solution of gallium-68. The specific activity in the spheres was 25.9 kBq/mL.

### EXAMPLES

### Example 1

Starting with an aqueous ⁶⁸Ge-solution in dilute hydrochloric acid, a phase transfer process was used to incorporate the germanium-68 into a hydrophobic acrylate-based monomer mixture for 3D printing. An 8 mL sphere was then printed with this radioactive building material in a stereolithography apparatus. After printing a wipe test was performed. The inventors checked source homogeneity with PET/CT imaging and by measuring pieces of the fragmented sphere in a gamma counter. Before fragmenting, the sphere was subjected to 50° C. ethanol for one hour to assess source tightness and isotope leaching.

### Material and Methods

### Chemicals and Isotopes

All chemicals and solvents were purchased from commercial suppliers. Tributylamine (nBu₃N, TBA; puriss. p.a., purity-GC 99.6%), lauryl gallate (dodecyl gallate; purity-HPLC 99.9%), n-butyl acrylate (purity-GC≥99%; stabilized with monomethyl ether hydroquinone, MEQH, 4-methoxyphenol), activated basic aluminum oxide (Al₂O₃, Brockmann I, particle size ˜150 mesh, pore size 58 Å), 0.1N hydrochloric acid, and 0.1N sodium hydroxide solution were from Sigma-Aldrich/Merck KGaA (Darmstadt, Germany), 2-propanol (≥99.8%, p.a.) from Carl Roth GmbH & Co.

KG (Karlsruhe, Germany), and 96% ethanol from Hänseler AG (Herisau, Switzerland). Germanium-68 in aqueous 0.05 M HCl was purchased from ITM Medical Isotopes GmbH

(Garching/München, Germany).

The ⁶⁸Ge-extraction experiments were performed in 1.5 mL or 50 mL polypropylene (PP) (micro) centrifuge tubes, respectively. A Spectrafuge™ 24D microcentrifuge (Labnet International, Inc.) was used for centrifugation. Weighting was performed on a calibrated XS205DU analytical balance from Mettler Toledo GmbH (Greifensee, Switzerland). For accurate radioactivity measurements, small aliquots of material (in quintuplet) were weighted on an analytical balance and counted for 2 min per sample (samples>1,000 cpm) or 1 h per sample (samples<1,000 cpm) in a 2470 Wizard2™ Automatic Gamma Counter (PerkinElmer, Waltham, MA, USA). All samples were measured 10 hours after sample preparation to ensure that the counts measured were representative of the ⁶⁸Ge-content of the sample, i.e., the gallium-68 present at the time of sample preparation had completely decayed (after 10 half-lives, >99.9% of initially present gallium-68 has decayed), and the secular equilibrium between the parent (⁶⁸Ge; t_{1/2},=271 days) and the daughter (⁶⁸Ga; t_{1/2},=68 min) had been established (after 7 half-lives, the daughter had reached >99% of the parent concentration).

Where applicable, counts-per-minute (cpm) values were converted to kBq by using a calibration curve based on a dilution series of samples with precisely known radioactivity concentration. For the preparation of the dilution series, the stock solution of germanium-68 in 0.05 M HCl from the manufacturer was transferred into a pre-weighted 11-mL-glass-vial (for this vial, the calibration factor in the ISOMED 2010 dose calibrator with different filling volumes had been determined previously), and the mass of the solution was determined on a microbalance. The 11-mL-glass-vial was sealed with a rubber septum, and the radioactivity of the solution was measured in the calibrated ISOMED 2010 dose calibrator from NUVIA Instruments GmbH (Dresden, Germany). At the time of the measurement, the mass of the total solution was 0.481 g and the radioactivity 43.85 MBq. Using a micropipette, ca. 10 μL of this original manufacturer's stock solution were transferred into a pre-weighted 1.5-mL microcentrifuge tube, and the precise mass of the solution was measured on a microbalance. The radioactivity transferred to the 1.5-mL microcentrifuge tube was then calculated based on the mass of the transferred solution and the known radioactivity concentration of the manufacturer's stock solution as determined in the dose calibrator. The ˜10 μL stock solution in the 1.5 mL microcentrifuge tube was then diluted with 0.05 M aqueous HCl to a radioactivity concentration of about 1 MBq/mL (the precise concentration was calculated based on the mass of the added 0.05 M HCl). This diluted stock solution was used to prepare two independent dilution series in 0.05 M aqueous HCl, with radioactivity concentrations of ˜20 kBq/g, ˜10 kBq/g, ˜4 kBq/g, ˜0.8 kBq/g, ˜0.16 kBq/g, ˜0.032 kBq/g (the precise concentration of each individual sample was calculated based on the masses of the solutions, which were determined on a microbalance). From these samples, 0.5 mL (exact mass for each sample determined on a microbalance) were transferred to Wizard tubes and counted in the 2470 Wizard2™ Automatic Gamma Counter. Based on these values, a cpm to Bq calibration curve was prepared. The correlation was linear throughout the entire radioactivity range tested.

### Procedure for Phase-Transfer of Germanium-68 into the Liquid Resin Monomer

Several preparatory steps were performed just before starting with the ⁶⁸Ge-extraction procedure: i) The radical stabilizer MEQH was removed from a small portion of n-butyl acrylate (10 mL) by passing the monomer through a column filled with activated basic aluminum oxide (length=3 cm, diameter=1 cm). ii) About 250 kBq of the ⁶⁸Ge-solution in 0.05 M HCl were transferred into a microcentrifuge tube, and the volume was adjusted to 100 μL by addition of the required volume of aqueous 0.05 M HCl. iii) Two reagent stock solutions were prepared in microcentrifuge tubes as follows: 200 mg/mL dodecyl gallate in 100% ethanol (stock solution I) and 120 mg/mL tributylamine in stabilizer-free n-butyl acrylate (stock solution II).

For the extraction of germanium-68, 20 μL of the ethanolic stock solution I (corresponding to 4 mg of dodecyl gallate) was added to the 100 μL of ⁶⁸Ge-solution in 0.05 M HCl, and the content was quickly mixed with a Vortex. Immediately thereafter, 100 μL of the organic stock solution II (corresponding to 12 mg of tributylamine, equal to ˜5.5 molar equivalent with respect to dodecyl gallate) was added, and the resulting biphasic mixture was thoroughly mixed on a Vortex for 1 min, followed by centrifugation for 3 min at 16300×g. The ⁶⁸Ge-containing organic upper layer was carefully transferred to a new, pre-weighted microcentrifuge tube by using a micropipette. Since care has to be taken not to contaminate the organic extracts with part of the water layer, minor losses due to a residual amount of the organic phase in the extraction tube were inevitable. Weighted aliquots of the organic layer and the water layer were counted in the gamma counter 10 hours after sample preparation to ensure that the gallium-68 measured in the gamma counter is solely derived from germanium-68 in the counting samples (c.f. above). The results were used to calculate the radioactivity concentration in each layer and to calculate the distribution coefficient and the extraction efficiency. Based on the desired radioactivity concentration in the final phantom, a suitable volume of the ⁶⁸Ge-containing organic extracts was carefully, but thoroughly mixed with the required volume of liquid monomer for printing. During this step, formation of air-bubbles and foam needed to be avoided. The radioactive liquid monomer was then transferred to the printer cartridge for printing. Weighted aliquots of the ⁶⁸Ge-containing liquid resin monomer (withdrawn during the transfer to the printer cartridge) were counted in the gamma counter to assess the homogeneity of the radioactivity distribution in the ⁶⁸Ge-containing liquid monomer.

TABLE 1
PHYSICAL PROPERTIES OF THE PRINTED SPHERES

| Nominal | Polar | Equatorial | | Nominal | Calculated | | Specific |
|---|---|---|---|---|---|---|---|
| diameter | diameter | diameter | Sphericity | Volume | volume | Weight | Weight |
| (mm) | (mm) | (mm) | (Ψ) | (ml) | (ml) | (g) | (g/ml) |
| 24.8 | n/a | n/a | | 8.00 | | 9.68 | 1.21± |
| 37.0 | 37.00 | 36.80 | 0.999995 | 26.52 | 26.21 | 30.81 | 1.18 |
| 28.0 | 27.92 | 27.80 | 0.999997 | 11.49 | 11.29 | 13.43 | 1.19 |
| 22.0 | 22.22 | 21.92 | 0.999967 | 5.75 | 5.58 | 6.58 | 1.18 |
| 17.0 | 17.20 | 16.94 | 0.999959 | 2.57 | 2.58 | 3.06 | 1.19 |
| 13.0 | 13.5 | 13.04 | 0.999854 | 1.15 | 1.20 | 1.40 | 1.17 |
| 10.0 | 10.12 | 9.98 | 0.999965 | 0.52 | 0.53 | 0.61 | 1.15 |
| 7.7 | 7.9 | 7.80 | 0.999970 | 0.24 | 0.25 | 0.30 | 1.20 |

Table 1: Planned (nominal) and measured physical properties of the printed spheres.
Sphericity was calculated according to (1) from measured polar and equatorial diameters.

### Stereolithography

3D printing of the sphere (diameter 25 mm, volume ˜8 mL) was performed on a ProJet® 1200 3D Printer (3D Systems, Inc., USA) operating with enhanced LED Digital Light Processing (DLP) technology. The net build volume of the ProJet® 1200 3D Printer was 43×27×150 mm³ (xyz), the native resolution (xyz) was 0.056 mm (effective 585 dpi), the layer thickness was 0.03 mm, and the vertical build speed was 14 mm/h [21]. The build material “VisiJet FTX Green” is a UV-curable liquid resin monomer mixture containing 40-55% of triethylene glycol diacrylate, 15-25% of tricyclodecane dimethanol diacrylate, and 1.5-2.5% of the photoinitiatorphenylbis (2,4,6-trimethylbenzoyl)phosphine oxide [22].

After completion of the stereolithography procedure, the printed object was rinsed twice with 2-propanol, dried, and then post cured in the UV curing chamber according to the instructions in the user guide [21].

Printing of a set of spheres ranging from 7.7 mm to 37 mm with germanium-68 (diameters 7.7/10.0/13.0/17.0/22.0/28.0/37.0 mm) was performed on a 3D printer Anycubic Photon Mono X using “Phrozen Resin ABS like Creamy White” as the build material.

The measured volumes, weights, and sphericity of the printed spheres are given in Table 1. Sphericity of all examined spheres was close to unity, as deviations from the target diameter were usually in the sub-millimeter range.

### Source Tightness Measurements

To detect potential leakage of radioactivity from the ⁶⁸Ge-phantom, we first conducted a wipe test using water-wetted and ethanol-wetted swabs (5 samples for each solvent), which were measured in the gamma counter (10 hours after sample preparation, vide supra). The limit of detection (LOD) and the limit of quantification (LOQ) in these tests were based on the standard deviation (SD) of the background samples (empty tubes with caps) and were calculated according to the expressions LOD=3×SD (background) and LOQ=10×SD (background). Using these expressions and a counting time of 1 h per sample, the LOD was 7 cpm, corresponding to 2.9 ppm of the total counts of the ⁶⁸Ge-phantom, and the LOQ was 22 cpm, corresponding to 9.5 ppm of the total phantom activity.

In order to ensure safe handling and long-term stability, the inventors also investigated potential leakage of germanium-68 from the phantom under accelerating conditions, i.e., elevated temperature, ethanol, acidic and basic aqueous extractions. To this end, the radioactive phantom was immersed in 96% ethanol (40 mL) in a closed 50 mL centrifuge tube, and the tube was heated in a water bath at 50° C. for 1 h. For optimal mixing, the tube was shaken several times during the extraction. After the first extraction, two additional ethanolic extractions were performed in the same manner with fresh ethanol (40 mL, 50° C., 1 h), followed by single extractions with 0.1 N aqueous HCl (40 mL, 50° C., 1 h) and 0.1 N aqueous NaOH (40 mL, 50° C., 1 h). Aliquots (1 mL) of the extraction solutions were measured in the gamma counter (within 10 hours after sample preparation, vide supra), and the extracted radioactivity in each extraction was expressed as a fraction of the total radioactivity of the ⁶⁸Ge-phantom.

### PET/CT Measurements

The printed 8 mL sphere or alternatively the liquid filled 8 mL sphere was mounted on a 120 mm long polymethylmethacrylate rod and positioned in the middle of a water-filled polymethylmethacrylate cylinder of 220 mm diameter and 186 mm length. The activity concentration of the filled sphere was 2.4 kBq/mL of germanium-68. The cylinder was then positioned with either the printed or the liquid filled sphere at the center of the PET/CT system's FOV. PET/CT data were acquired for 18.4 h for the liquid filled sphere and 15 h for the printed sphere to obtain very low noise PET images. Both PET measurements were corrected for decay, attenuation, randoms, dead-time and scatter. Images were then reconstructed into an 880×880×880 matrix with filtered backprojection (FBP) or ordered subset expectation maximization (OSEM). All reconstruction were done with 1 mm Gaussian post-reconstruction filtering. Image slice thickness was 0.8 mm, producing isometric voxels. Image data were stored and handled in the DICOM format. Sphere images were analyzed within a cuboid of 120 voxels edge length centered onto the sphere centroid using IGRO Pro 8 (WaveMetrics, Lake Oswego, OR, USA).

For PET attenuation correction and for the analysis of printing material homogeneity, we performed CT scans with 120 keV tube voltage, 80 mAs tube current, 1.5 mm collimation slice thickness and 0.8 pitch. The CT images were reconstructed into a 512×512 matrix.

### Destructive Analysis of the 3D-Printed ⁶⁸Ge-Phantom

The printed 8 mL sphere was immersed in liquid nitrogen (-−96° C.), quickly placed in a thick transparent plastic bag, and destroyed using a hammer. The pieces were transferred into Wizard tubes (10 tubes; ca. 40-200 mg/tube) and counted in the 2470 Wizard2™ Automatic Gamma Counter. The cpm values were used to calculate the radioactivity concentration in kBq/g and kBq/mL using the cpm-kBq calibration curve and the measured mass (9.747 g) and the measured volume of the sphere (8.029 mL). Additionally, the homogeneity of the radioactivity distribution in the printed ⁶⁸Ge-phantom was assessed by determining the relative standard deviation (RSD) of the radioactivity concentration of the 10 samples with the fragments.

### Results

### Complexation and Phase-Transfer of Germanium-68

To ensure a safe working procedure and stable incorporation of germanium-68 into the 3D-printed solid phantom, several key challenges needed to be addressed. Firstly, appropriate measures have to be taken to avoid formation and release of volatile radioactive germanium halides during the manufacture as well as during the whole lifetime of the 3D-printed solid phantom. Secondly, the radioactive germanium-68 needs to be transferred from the commercially available dilute aqueous hydrochloric acid solution into the organic liquid 3D building material, which is not miscible with water. The inventors aimed to solve both issues simultaneously by developing a combined complexation-extraction procedure based on chelation of germanium-68 with dodecyl gallate followed by tributylamine-induced phase transfer from the aqueous to the organic layer, which is then mixed with the organic liquid resin monomers, i.e. the 3D building material.

Initial experiments confirmed that the extraction of germanium-68 requires the presence of both the complexing agent dodecyl gallate as well as the phase transfer agent tributylamine, since efficient transfer could not be achieved in the absence of one of these agents. The inventors continued by evaluating the effect of different concentrations of both reagents, solvent volume, and other extraction parameters. The optimized procedure involves mixing of the ⁶⁸Ge-solution in hydrochloric acid with an ethanolic stock solution of dodecyl gallate, followed by addition of a stock solution of tributylamine in n-butyl acrylate, repeated mixing, centrifugation, and finally removal of the ⁶⁸Ge-containing organic upper layer into a clean microcentrifuge tube.

The potency of the extraction procedure was assessed by determining the radioactivity concentration of weighted aliquots from both phases >10 h after preparation to ensure that the equilibrium between the parent germanium-68 and the daughter gallium-68 had been established in each phase. The observed radioactivity concentration ratio between the organic versus the aqueous phase was >3700:1, which renders this procedure very efficient. Depending on the desired radioactivity concentration in the object to be printed, suitable volumes of the ⁶⁸Ge-containing n-butyl acrylate phase and the organic liquid 3D building material were thoroughly mixed and transferred to the printer cartridge for 3D printing. The target activity concentration was 10-11 kBq/mL as confirmed by PET/CT measurements. The homogeneity of the radioactivity distribution in the liquid 3D building material was confirmed by determining the radioactivity concentration of several aliquots of the material withdrawn during filling of the printer cartridge. The relative standard deviation (RSD) of the radioactivity concentration of the samples was <2% (n=5).

### Stereolithography and Source Tightness Measurements

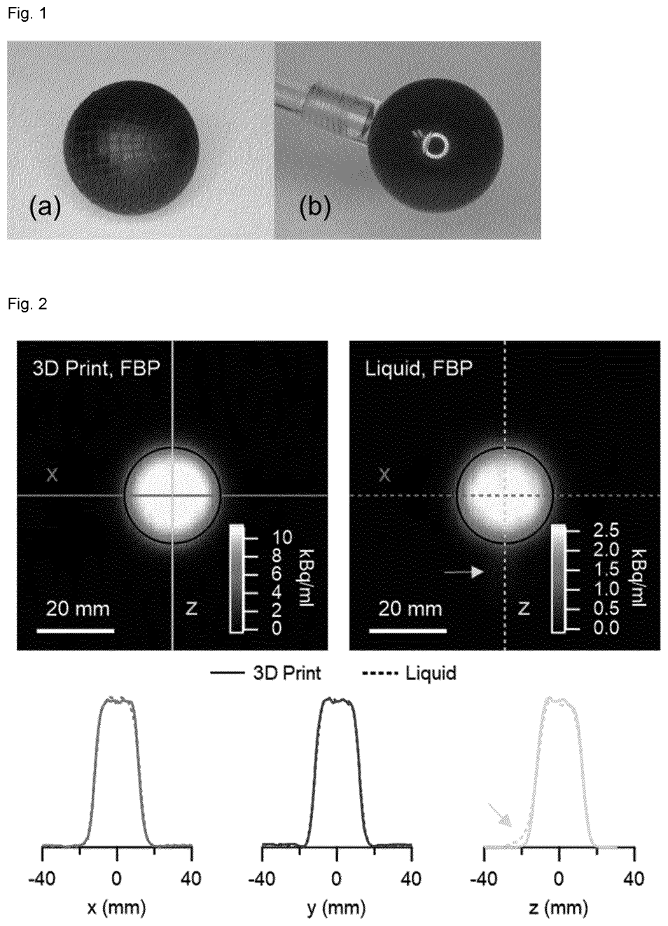

Stereolithography with the ⁶⁸Ge-containing building material was performed in the same way as with the original printer cartridges filled with non-radioactive liquid monomer resin. The inventors did not note any differences in printing behaviour. Also regarding the further processing—rinsing, drying, and post curing—the inventors did not deviate from the standard procedures used for 3D printing with the non-radioactive building material. FIG. 1 depicts the final printed sphere in comparison with the liquid filled sphere.

The source tightness of the ⁶⁸Ge-containing sphere was assessed by performing wipe tests with water-wetted and ethanol-wetted swabs. Both wipe tests yielded results marginally above the limit of detection (LOD, corresponding to 2.9 ppm of the total counts of the phantom), but well below the limit of quantification (LOQ; corresponding to 9.5 ppm of the total activity of the phantom). In order to quantify the surface-extractable radioactivity and to obtain more insight into the long-term stability of the phantom, the leakage of germanium-68 was investigated under accelerating conditions, i.e., elevated temperature, ethanolic, and aqueous acidic as well as aqueous basic extractions. First, three sequential extractions were performed in 96% ethanol at 50° C. (1 h per extraction). The extractable radioactivity (expressed as fraction of the total radioactivity of the phantom) dropped successively from 1.8% o in the first extraction to 0.7% in the second, and to 0.5% in the third extraction. The subsequent acidic and basic extractions at 50° C. in 0.1 N aqueous HCl and 0.1 N aqueous NaOH, respectively, yielded extractable radioactivity values just above the limit of detection, but far below the limit of quantification (LOQ=0.4%).

### PET/CT Measurements

FIG. 2 shows the PET/CT images of the printed and liquid filled 8 mL spheres reconstructed with FBP. Visually, almost no differences can be discerned between the printed and the filled sphere, except that in the filled sphere image the filler neck can be recognized in axial (z) direction. The line profiles running through the centroid of the sphere in x- and y-direction directions are indistinguishable between the printed and the filled spheres. The z-line profile reveals residual activity in the filler neck of the filled sphere (FIG. 2, green arrow).

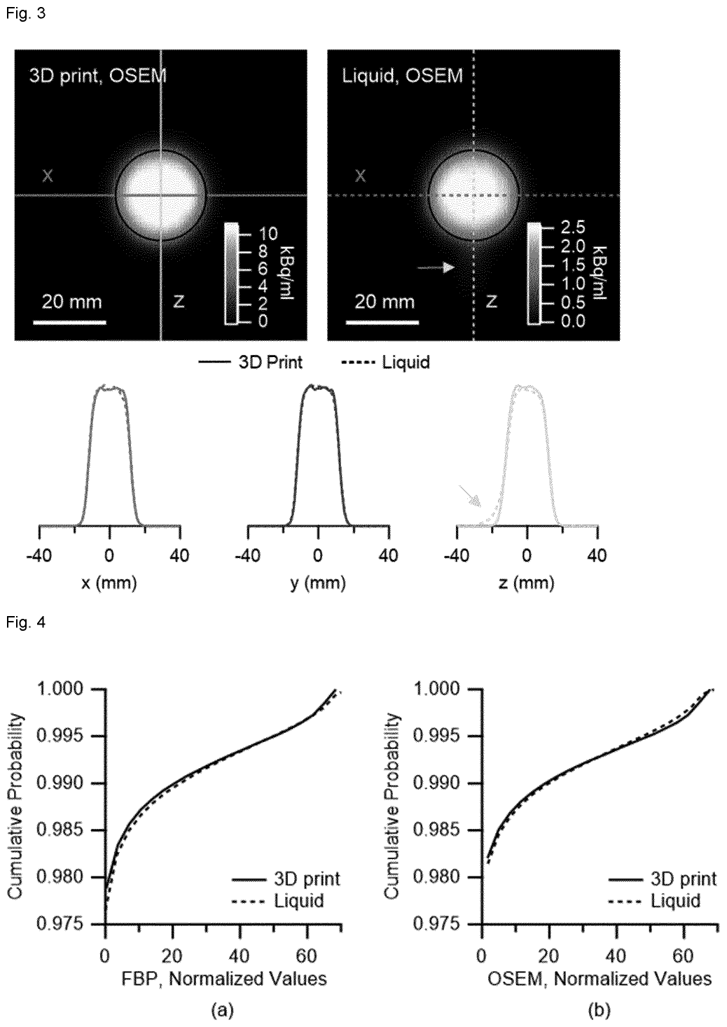

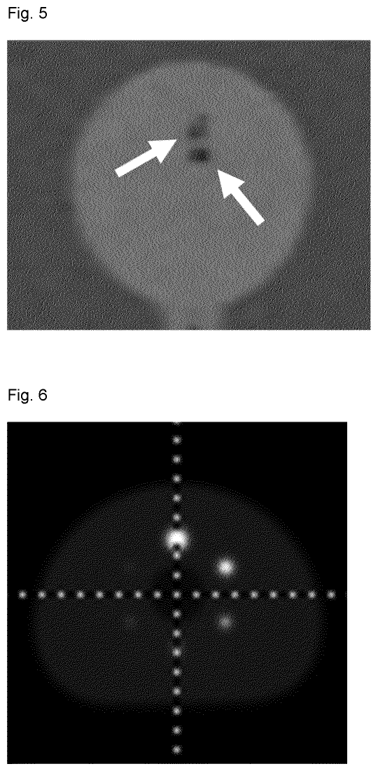

FIG. 3 shows the same for OSEM images. The slightly better spatial resolution of OSEM reconstruction makes the filler neck signal even more visible. In the printed spheres, the maximal recorded value was 11.3 kBq for the FBP reconstruction and 11.2 kBq/mL for the OSEM reconstruction. The measured activity at the printed sphere centroid was 10.8 kBq/mL for both FBP and OSEM reconstructions.

Cumulative histograms calculated form the total activity found within a cuboid of 120×120×120 voxels around the sphere centroid show almost no difference in activity distribution between the printed and filled sphere regardless of image reconstruction (FIG. 4).

High-resolution CT scans revealed three voids within the printed sphere, with the largest void having a diameter of less than 1 mm.

The same voids were also observed in non-radioactive printed spheres of equal diameter. Smaller printed spheres did not reveal such artefacts. We conclude that the defects were not a consequence of our phase transfer method but are connected to the printing.

In another setup the germanium-68 phantom consisting of 6 spheres were measured in a decaying gallium-68 solution to see the influence of different foreground to backgrounds in a state of the art PET/CT scanner Biograph Vision Quadra. The phantom was set up according to the published specifications of NEMA NU 2-2018 Standard except for the biggest sphere of 37 mm in diameter, which was replaced against a 7.7 mm sphere to consider the very significant advances of imaging compared to the time, when the standard was set. Such a setup offers testing machines, and reconstruction algorithms with phantoms of known activity and varying backgrounds in short time in order to optimize for high resolution, low noise and quantitative results.

### Validation of PET-Data by Destructive Analysis of the 3D-Printed 68Ge-Phantom

To verify the radioactivity concentration data derived from the PET-scans, the 3D-printed sphere was destroyed and the radioactivity of the weighted fragments was determined in a Gamma Counter. The radioactivity concentration based on the destructive analysis was 10.9±0.3 kBq/mL (Mean±SD), essentially identical to the value calculated from the PET-data. Furthermore, the relative standard deviation (RSD) of the radioactivity concentration of the phantom-fragments in the destructive analysis was <2% (n=10), confirming that the radioactivity was homogeneously distributed throughout the 3D-printed ⁶⁸Ge-phantom.

### CONCLUSION

The inventors successfully transferred germanium-68 into the printing monomer and demonstrated that manufacturing hot spheres without cold walls is possible with this building material. After printing, the resulting 8 mL sphere had a uniform radioactivity distribution of 10.9±0.3 kBq/mL with near perfect isotope retention. The radioactivity was homogeneously distributed and PET images of the printed sphere were indistinguishable from PET images of a liquid filled 8 mL sphere. Actually, the liquid filled sphere retained residual activity in the filler neck, something that can be definitely ruled out in the printed sphere.

Not only is the combined chelating and phase transfer method essential for a homogeneous final product wherein the germanium-68 is stably immobilized during the whole lifetime of the phantom, but it is inherently safer in terms of radiation protection than a simple mixture of resin and aqueous ⁶⁸Ge-solutions ever could be. The energetically favorable process provides complexation and phase transfer of germanium-68 into the hydrophobic building material, ensures homogenous isotope distribution in the rigid polymer matrix and prevents leaching in aqueous environment. The strong isotope retention makes cold walls obsolete for radiation safety. This avoids fringe effects at foreground-background interfaces [17] and gives a better physiological representation. Furthermore, the inventive method has successfully been tested with other commercially available printing monomers that work with a different type of stereolithographic apparatus.

Taken together, the inventors have developed a combined complexation-extraction procedure to solve two key challenges, which have so far hampered the 3D-printing of ⁶⁸Ge-based PET phantoms, namely the transfer of germanium-68 from an aqueous solution into an organic acrylate-based monomer and the safe containment of germanium-68 in the monomer the form of non-volatile hydrophobic complexes. Said procedure maximizes germanium-68 transfer into the monomer and allows for a precise control of the final activity concentration. With the resulting radioactive monomer, rapid, easy and decentralized manufacturing of phantoms for molecular imaging becomes feasible. The readily available aqueous germanium-68 solution makes the inventive method inherently suited for low-cost manufacturing of long-lived PET/CT phantoms of arbitrary shape. Furthermore, the inventive method is adaptable to other 3D-shaping polymer-based processes, different polymers and other radionuclides.

### Example 2

This example describes the procedures and results of representative phase transfer experiments with the radionuclides cobalt-57 and lutetium-177, using the methods claimed in this patent.

### Procedure for Phase-Transfer of Cobalt-57 and Lutetium-177 to Butyl Acrylate with Dodecyl Gallate

To transfer a radioisotope from an aqueous phase directly to butyl acrylate or butyl acetate, an aqueous radioisotope solution of known specific activity is used. In a typical experiment, 130 μL of butyl acrylate as a monomer or organic solvent, 5 mg of dodecyl gallate as the complex-forming lipophilic ligand, and 15 μL of tributylamine as an additive are mixed in a 1.5 mL Eppendorf tube. Then 150 μL of the radioisotope solution are added. The samples are mixed in a shaker at RT for 30 minutes, and centrifuged. The phases are then separated and used for either determination of the distribution coefficient or for radiolabeling.

Distribution coefficient calculation: To determine the distribution coefficient, 50 μL of each phase are separated using an adjustable pipette and the solution activities of both phases are measured in a Perkin-Elmer Wizard gamma counter. The distribution coefficient is calculated as the ratio of the determined counts in the organic phase to the counts in the aqueous phase.

We tested the gamma emitter cobalt-57 with a half-life of 272d. An aqueous solution of cobalt-57 chloride with a specific activity of about 1.5 MBq/mL was used.

After the phase separation procedure, the cobalt-57 distribution coefficient was determined to be 71:1. As a reference, we tested the influence of all additives separately and calculated the distribution coefficient. The determined ratio of the counts in the organic phase to the counts in the aqueous phase with only butyl acrylate was 1:40,000, with 5 mg lauryl gallate in butyl acrylate, it was 1:34,000, and with 15 μL triethylamine in butyl acrylate, it was 1 5,600.

The results clearly show that both lauryl gallate and triethylamine are required to achieve efficient transfer of cobalt-57 to the organic phase.

We also determined the phase distribution of the electron and gamma emitter lutetium-177 with a half-life of 6.7 d. An aqueous solution of lutetium-177 chloride with a specific activity of about 5 MBq/mL was used. The distribution coefficient of butyl acrylate to water was 20.8:1. The partition coefficient with butyl acrylate only without additives was 1:130, with lauryl gallate only in butyl acrylate, it was 1:110, and with tributylamine only in butyl acrylate, it was 1:17,800.

For printing, the organic phase can be directly added to the monomer solutions for 3D printing and well mixed.

### Procedure for Phase-Transfer of Cobalt-57 to Butyl Acrylate with 8-Hydroxy-Chinolin

In a 1.5 mL Eppendorf tube, 10 mg of 8-hydroxy-chinolin as the complex-forming lipophilic ligand were dissolved in 150 μL butyl acrylate as a monomer or organic solvent. Then 150 μL of cobalt-57 solution in 0.1 mol/L ammonium acetate are added. The samples are mixed in a shaker at 80° C. for 30 minutes, and centrifuged. The phases are then separated and used for either determination of the distribution coefficient or for radiolabeling. A distribution of 41.1:1 was calculated for cobalt-57.

### REFERENCES

[1] I. Hristova, R. Boellaard, P. Galette, L. K. Shankar, Y. Liu, S. Stroobants, et al., “Guidelines for quality control of PET/CT scans in a multicenter clinical study,” EJNMMI Physics, vol. 4, p. 23, Sep. 18, 2017.
[2] Federal Office of Public Health, “Weisung L-09-04: Qualitätssicherung bei Gamma-, PET-, PET-CT-Kameras,” in Weisung L-09-04, F. D. o. H. Affairs, Ed., 1 ed. Bern, Switzerland: Swiss Confederation, 2018, p. 6.
[3] NEMA Standards Publication NU 2-1994: Performance Measurements of Positron Emission Tomographs, N. E. M. Association, 1994.
[4] NEMA Standards Publication NU 2-2018: Performance Measurements of Positron Emission Tomographs (PET), N. E. M. Association, 2018.
[5] R. Boellaard, R. Delgado-Bolton, W. J. G. Oyen, F. Giammarile, K. Tatsch, W. Eschner, et al., “FDG PET/CT: EANM procedure guidelines for tumour imaging: version 2.0,” Eur J Nucl Med Mol Imaging, vol. 42, pp. 328-354, 2015.
[6] N. Aide, C. Lasnon, P. Veit-Haibach, T. Sera, B. Sattler, and R. Boellaard, “EANM/EARL harmonization strategies in PET quantification: from daily practice to multicentre oncological studies,” Eur J Nucl Med Mol Imaging, vol. 44, pp. 17-31, August 2017.
[7] P. Kinahan, P. Christian, J. Karp, C. Divgi, G. Zubal, V. Pappas, et al., “A calibration phantom for multi-center PET/CT studies of assessing response to therapy,” Journal of Nuclear Medicine Meeting Abstracts, vol. 48, pp. 194P-b-, May 1, 2007.
[8] C. M. Lockhart, L. R. MacDonald, A. M. Alessio, W. A. McDougald, R. K. Doot, and P. E. Kinahan, “Quantifying and reducing the effect of calibration error on variability of PET/CT standardized uptake value measurements,” J Nucl Med., vol. 52, pp. 218-24, February 2011.
[9] P. Kinahan, R. Doot, P. Christian, J. Karp, J. Scheuermann, R. Zimmerman, et al., “Multi-center comparison of a PET/CT calibration phantom for imaging trials,” Journal of Nuclear Medicine Meeting Abstracts, vol. 49, pp. 63P-, May 1, 2008.
[10] R. K. Doot, J. S. Scheuermann, P. E. Christian, J. S. Karp, and P. E. Kinahan, “Instrumentation factors affecting variance and bias of quantifying tracer uptake with PET/CT,” Med. Phys., vol. 37, pp. 6035-46, November 2010.
[11] Internation Atomic Energy Agency, Regulations for the Safe Transport of Radioactive Material. Vienna International Centre, Vienna: International Atomic Energy Agency, 2018.
[12] J. I. Gear, C. Long, D. Rushforth, S. J. Chittenden, C. Cummings, and G. D. Flux, “Development of patient-specific molecular imaging phantoms using a 3D printer,” Med. Phys., vol. 41, p. 082502, 2014.
[13] G. A. Prenosil, B. Klaeser, M. Hentschel, M. Fürstner, M. Berndt, T. Krause, et al., “Isotope independent determination of PET/CT modulation transfer functions from phantom measurements on spheres,” Med. Phys., vol. 43, pp. 5767-5778, 30 Sep. 2016.
[14] G. A. Prenosil, T. Weitzel, M. Hentschel, B. Klaeser, and T. Krause, “Transconvolution and the virtual positron emission tomograph—A new method for cross calibration in quantitative PET/CT imaging,” Med. Phys., vol. 40, p. 15, 2013.
[15] E. Quak, P.-Y. Le Roux, M. Hofman, P. Robin, D. Bourhis, J. Callahan, et al., “Harmonizing FDG PET quantification while maintaining optimal lesion detection: prospective multicentre validation in 517 oncology patients,” Eur J Nucl Med Mol Imaging, pp. 1-11, 2015.
[16] J. I. Gear, C. Cummings, J. Sullivan, N. Cooper-Rayner, P. Downs, I. Murray, et al., “Radioactive 3D printing for the production of molecular imaging phantoms,” Phys Med Biol., vol. 65, p. 175019, Sep. 8, 2020.
[17] F. Hofheinz, S. Dittrich, C. Potzsch, and J. Hoff, “Effects of cold sphere walls in PET phantom measurements on the volume reproducing threshold,” Phys Med Biol., vol. 55, pp. 1099-113, Feb. 21, 2010.
[18] F. H. Fahey, P. E. Kinahan, R. K. Doot, M. Kocak, H. Thurston, and T. Y. Poussaint, “Variability in PET quantitation within a multicenter consortium,” Med. Phys., vol. 37, pp. 3660-6, July 2010.
[19] G. Klopman, J.-Y. Li, S. Wang, and M. Dimayuga, “Computer Automated log P Calculations Based on an Extended Group Contribution Approach,” Journal of Chemical Information and Computer Sciences, vol. 34, pp. 752-781, 1994.
[20] L. Xing and R. C. Glen, “Novel Methods for the Prediction of logP, pKa, and logD,” Journal of Chemical Information and Computer Sciences, vol. 42, pp. 796-805, 2002.
[21] 3D Systems, Inc., (2014 April). ProJet™ 1200 Professional 3D Printer User Guide (P/N 341410-00 Rev. F ed.). Available: http://infocenter.3dsystems.com/product-library/sites/default/files/printers/projet1200/2515_341378-00_RevF_UserGuide.pdf
[22] 3D Systems, Inc., (2019). Safety Data Sheet VisiJet® FTX Green. Available: http://infocenter.3dsystems.com/materials/sites/default/files/sds-files/professional/VisiJet_FTX/341600-S12-02-A%2CSDS%20GHS%2CGerman%2CVisiJet%20FTX%20Green.pdf
[23] T. Läppchen, L.P. Meier, M. Fürstner, G.A. Prenosil, T. Krause, A. Rominger, B. Klaeser, M. Hentschel, et al., “3D printing of radioactive phantoms for nuclear medicine imaging,” EJNMMI Physics, 7:22

1. A method for preparing a radioactive polymerizable solution comprising the steps of
a) providing
an aqueous radionuclide solution comprising a cationic radionuclide with a half-life >5 d,
a complex-forming lipophilic ligand, wherein
the complex forming lipophilic ligand comprises one or more carbon atoms and/or one or more silicon atoms, and one or more heteroatoms, wherein at least one heteroatom is selected from oxygen, nitrogen, sulfur, phosphorus, selenium and arsenic, and wherein the ratio of carbon atoms and/or silicon atoms to heteroatoms is ≥2:1, particularly ≥2.5:1, more particularly ≥3:1, wherein the heteroatoms that are considered for the ratio are selected from oxygen, nitrogen, sulfur, phosphorus, selenium and arsenic, and
the mass of the complex-forming lipophilic ligand is <5000 g/mol, particularly <1500 g/mol,
optionally, an additive, wherein the additive is a base or a suitable cation or anion acting as an auxiliary ligand and/or for charge compensation,
a monomer solution comprising one or more monomers selected from an acrylate and a methacrylate, wherein the monomer solution is non-miscible with said aqueous radionuclide solution,
optionally, an organic solvent,
b) mixing the aqueous radionuclide solution with
the complex forming lipophilic ligand, and
optionally the additive, and
the monomer solution or the organic solvent yielding an aqueous phase and a radioactive organic phase,
c) separating and collecting the radioactive organic phase in case of using the monomer solution in step (b), or separating and collecting the radioactive organic phase in case of using the organic solvent in step (b) and adding said monomer solution to the radioactive organic phase.

2. The method according to claim 1, wherein the cationic radionuclide is a cation of Ge, Na, Co or Lu, particularly ⁶⁸Ge, ²²Na, ⁵⁷Co or ¹⁷⁷Lu.

3. The method according to claim 1, wherein the distribution coefficient of the radiometal-complex between the organic phase and the water phase, under the conditions of the extraction and if applicable in the presence of the additive, is at least >5:1.

4. The method according to claim 1, wherein the complex-forming lipophilic ligand is selected from
a gallate, particularly an alkyl gallate,
pyrocatechol,
a di- or tricarboxylic acid,
tannin or a derivative thereof,
8-hydroxyquinoline or a derivative thereof,
a crown-ether, a cryptand, a podand, a spherand, a calixarene, or a derivative thereof,
trialkylphosphine,
a thiol, a thioether, or a derivative thereof,
mono-, di-, or tri-alkyl-DTPA or derivatives thereof containing aryl, heteroaryl and/or other functionalities at any position in one or more of the alkyl chains,
mono-, di-, and tri-alkyl-DOTA or derivatives thereof containing aryl, heteroaryl and/or other functionalities at any position in one or more of the alkyl chains, in particular from
a gallate, particularly an alkyl gallate,
pyrocatechol,
a di- or tricarboxylic acid,
tannin or a derivative thereof,
8-hydroxyquinoline or a derivative thereof,
a crown-ether, a cryptand, a podand, a spherand, a calixarene, or a derivative thereof,
a thiol, a thioether, or a derivative thereof,
mono-, di-, or tri-alkyl-DTPA or derivatives thereof containing aryl, heteroaryl and/or other functionalities at any position in one or more of the alkyl chains,
mono-, di-, and tri-alkyl-DOTA or derivatives thereof containing aryl, heteroaryl and/or other functionalities at any position in one or more of the alkyl chains.

5. The method according to claim 1, wherein the complex forming ligand is substituted by one or more acrylate moieties and/or one or more methacrylate moieties.

6. The method according to claim 1, wherein the additive is a hydrophobic amine, particularly a mono-, di-, or trialkylamine, the corresponding tetraalkylammonium salts, a triflate, a mesylate, a tosylate, a benzoate, a salicylate, a perchlorate, a tetrafluoroborate, tetrafluoro carboxylate, an alkyl sulfonate, an alkyl phosphonate.

7. The method according to claim 1, wherein the additive is used in step (b) if the radionuclide is a cation of ⁶⁸Ge.

8. The method according to claim 1, wherein
the acrylate of the monomer solution is selected from a mono-acrylate, a di-acrylate and a tri-acrylate, particularly tricyclodecane dimethanol diacrylate, mono-, di-, or tri-ethylene glycol diacrylate, bisphenol A ethoxylate diacrylate, and/or
the methacrylate of the monomer solution is selected from a mono-methacrylate, a di-methacrylate and a tri-methacrylate, particularly tricyclodecane dimethanol di(meth)acrylate, mono-, di-, or tri-ethylene glycol di(meth)acrylate, bisphenol A ethoxylate dimethacrylate.

9. The method according to claim 1, wherein the organic solvent is selected from an acrylate, a methacrylate, and acetate or a mixture thereof, particularly from an alkylacrylate, alkylmethacrylate and alkylacetate or a mixture thereof.

10. A radioactive polymerizable solution, particularly prepared according to the method according to claim 1, comprising a complex dissolved in a monomer solution comprising one or more monomers selected from an acrylate and a methacrylate, wherein the complex comprises
a cationic radionuclide with a half-life >5 d, and
a complex-forming lipophilic ligand, wherein
the complex forming lipophilic ligand comprises one or more carbon atoms and/or one or more silicon atoms, and one or more heteroatoms, wherein at least one heteroatom is selected from oxygen, nitrogen, sulfur, phosphorus, selenium and arsenic, and wherein the ratio of carbon atoms and/or silicon atoms to heteroatoms is ≥2:1, particularly ≥2.5:1, more particularly ≥3:1, wherein the heteroatoms that are considered for the ratio are selected from oxygen, nitrogen, sulfur, phosphorus, selenium and arsenic, and
the mass of the complex-forming lipophilic ligand is <5000 g/mol, particularly <1500 g/mol,
optionally an additive, wherein the additive is a base or a suitable cation or anion acting as an auxiliary ligand and/or for charge compensation.

11. The radioactive polymerizable solution according to claim 10, wherein the radiolabeled printing solution further comprises an initiator, particularly a photoinitiator, more particularly phenylbis (2,4,6-trimethylbenzoyl)phosphine oxide.

12. A method for preparing a radioactive object, comprising the steps of
a) providing a radioactive polymerizable solution prepared according to the method according to claim 1,
b) adding an initiator, particularly a photoinitiator, more particularly phenylbis(2,4,6-trimethylbenzoyl)phosphine oxide
c) polymerization by using light or UV light.

13. A radioactive object, particularly prepared according to the method according to claim 12, comprising a complex distributed in a polymer network comprising one or more polymers selected from an acrylate polymer and a methacrylate polymer, wherein the complex comprises
a cationic radionuclide with a half-life >5 d, and
a complex-forming lipophilic ligand, wherein
the complex-forming lipophilic ligand comprises one or more carbon atoms and/or one or more silicon atoms, and one or more heteroatoms, wherein at least one heteroatom is selected from oxygen, nitrogen, sulfur, phosphorus, selenium and arsenic, and wherein the ratio of carbon atoms and/or silicon atoms to heteroatoms is ≥2:1, particularly ≥2.5:1, more particularly ≥3:1, wherein the heteroatoms that are considered for the ratio are selected from oxygen, nitrogen, sulfur, phosphorus, selenium and arsenic, and
the mass of the complex-forming lipophilic ligand is <5000 g/mol, particularly <1500 g/mol,
optionally an additive, wherein the additive is a base or a suitable cation or anion acting as an auxiliary ligand and/or for charge compensation.

14. The radioactive object according to claim 13, wherein the radioactive object is a phantom for quantitative positron emission tomography (PET) or/and PET/CT and/or quantitative single emission computed tomography (SPECT) or/and SPECT/CT, or/and other devices for the quantitative detection of radioactivity.

15. The radioactive object according to claim 13, wherein the radioactive object represents an organ, tumor, another body part or combinations thereof.
